(19) United States
(12) Patent Application Publication
(10) Pub. No.: US 2018/0008489 A1
(43) Pub. Date: Jan. 11, 2018

(54) Discreet Emergency Hygiene Kit

(71) Applicants: Jewel Victoria Miles, Snellville, GA (US); Alen Dino Kalac, Talmo, GA (US)

(72) Inventors: Jewel Victoria Miles, Snellville, GA (US); Alen Dino Kalac, Talmo, GA (US)

(21) Appl. No.: 15/206,232

(22) Filed: Jul. 9, 2016

(51) Int. Cl. A61F 13/551; B65D 85/18; B65D 75/30; B65B 47/02; B65B 51/10; A61F 6/00; B65B 25/20; B65B 7/02; B65D 85/00; B65B 63/04

(52) U.S. Cl. CPC A61F 13/5519; B65D 85/70; B65D 85/18; B65D 75/30; B65B 63/045; B65B 51/10; A61F 6/005; B65B 25/20; B65B 7/02; A61F 13/55175; A61F 13/55145; B65B 47/02

(57) ABSTRACT

A discreet packaged sterile hygiene kit for the unexpected menstrual cycle and safe sex. Kit is comprised such as at least sterile scented underwear, or leggings, and either one in addition to the undergarments/leggings and at least tampons, sanitary pads, panty liners, and at least sterile wipes, and at least prophylactic birth control/safe sex. Packaged uniquely in a small confined vacuum packed plastics packaging which fits inconspicuously inside a purse, backpack, locker, desk, vehicle glove box, or any place of choosing; thus allowing its user maximum and or ultimate privacy. Underwear is folded tightly then inserted into the packaging in one pocket followed by the insertion of either tampons, sanitary pads, panty liners, wipes or prophylactic into another pocket. Extra items being provided in its own packaging material. Package is sealed and packaged in a manner that will allow displaying via; vending machines, store racks, or any retail display.

### BACKGROUND OF THE INVENTION

The present invention is a utilitarian product that is a conveniently packaged emergency kit for females of various ages to ensure that the necessary clean sterile undergarment and feminine hygiene items such as, tampons, sanitary napkins panty liners, and wipes are on hand when the unexpected menstrual happens and a kit for male or female with condom and sterile undergarment for safe sex.

The design and discreet packaging allows females of all ages to be prepared when the unexpected menstrual cycle happens or leakage. Gives women of various ages the piece of mind and worry free of embarrassment if it falls out of their purse, back pack, locker, desk, car, or any place of storage into public view.

It is necessary for females to have on hand or availability of feminine hygiene products such as tampons, sanitary napkins, panty liners, wipes and clean undergarments because females never really know the exact day their period will start.

This product is absolutely inconspicuous, because of it's packaging. If the product falls out into public view and someone sees it, it will spare them of the embarrassment. Can be kelp in clear view, stored in such as locker, desk, gym, purse, backpack, car any storage of personal choice.

This invention is ideal to keep on hand for the unexpected period, because females just never really know when will be the onset of her period. Therefore, this is a great solution for females of various ages for their period or leakage and/or safe sex. They find themselves in a situation where they do not have the needed supplies such as sterile clean undergarments, feminine hygiene products and/or wipes and/or condom with clean underwear for safe sex. This invention is a needed product for the above issues and is an effective solution to the problem in a discreet manner.

### FIELD OF THE INVENTION

The present invention relates generally to a package hygiene/safe sex kit that is discreetly and inconspicuously and more particularly to a package for absorbent hygiene products and condoms and at least undergarments.

The package contains at least one undergarment and at least one hygiene product or condom. The present invention allows for women/male to discreetly be prepared for the unexpected period and prepared for safe sex.

### SUMMARY

The novel inventions is a product, which fills a void in the marketplace. The invention is an assembly, which uses other products within it.

The novel intention includes a package design that accommodates discreetly a sterile pair of undergarments or leggings and either one items such as tampons, sanitary pads, panty liners, and wipe or such as a condom.

The kit is designed to meet the needs of an unexpected menstrual cycle while in public and/or away from home. When the unexpected period happens and comes without warning, it is desirable to have some type of feminine hygiene item or product readily available.

Absolutely inconspicuous, because of its packaging. If the product falls out in public and someone sees it, it will spare them of embarrassment.

Packaged discreetly and conveniently for easy carry; in a purse, desk drawer, locker, glove box, backpack and/or such as any discreet place of your choosing.

This invention is a needed product for the above issues and is an effective solution to the problem in a discreet manner.

### BRIEF DESCRIPTION OF THE FIGURES

FIG. 1

Is a front view of the present invention.

FIG. 2

Is a side view of the present invention.

FIG. 3

Is a front view of the present invention.

FIG. 4

Is a charcoal rendition of packaged product.

FIG. 5

Is an actual finished packaged product of present invention showing the product that is inside the package.

### DETAILED DESCRIPTION OF THE INVENTION DRAWINGS

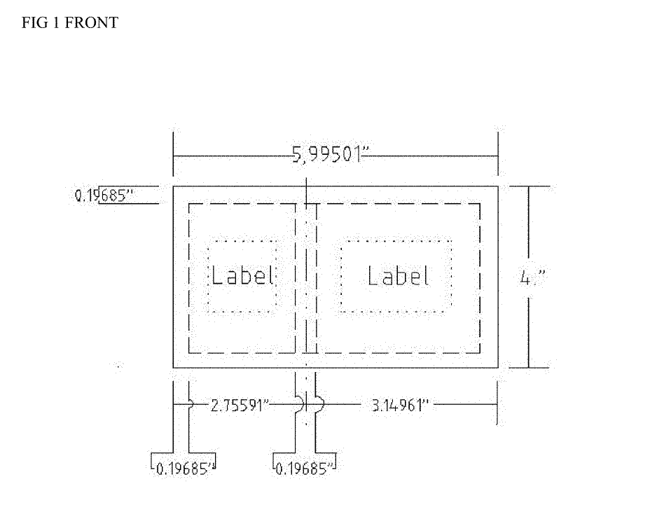

FIG. 1 Front

The top view drawing depicts the packaging from top view measuring approximately 4″×5.99501″ inches in total H×L, indicating H×L, where H equals to height and L equals to length.

The packaging is heat sealed along the perimeter with a 0.19501 “less than approximately quarter inch” inch seam through out its edge.

This measure is the surface contact width of each side panel used for making the packaging.

The sealed products consisting of a vacuum packed panty with a choice of; pad, tampon, or condoms is “compartmentalized” individually with a “center division” consisting of a heat seal measuring approximately 0.19501 inches in width, adjoining both sides of the packaging.

The center of such center seal seam is approximately 2.75591″ from the left side edge of the packaging and extends approximately 3.14961″ inches from the off center of the packaging to the outer right seal edge.

Note;

The top view “artist rendition” depicts the aforementioned and includes colorful flowery design and product information label with a note describing the dotted lines indicating seal.

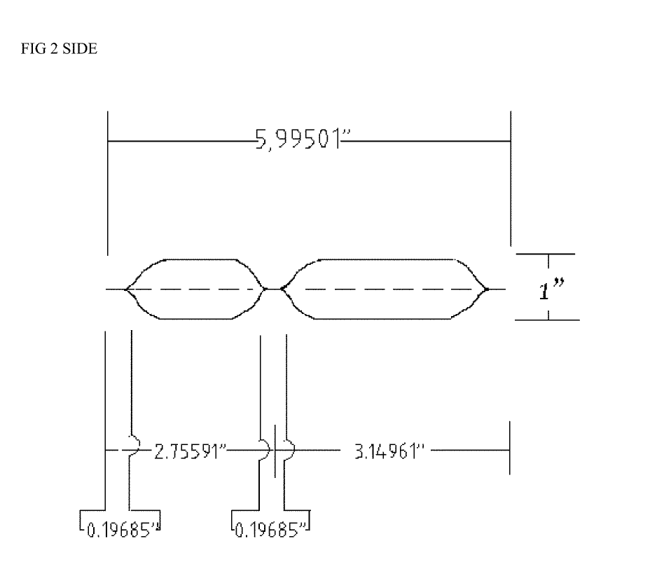

FIG. 2 Side Left and Right

FIG. 2 depicts a product from is peripheral view, “inferring on a flat surface.”

The product vacuum packaging is depicted from length view side measuring approximately 5.99501″ inches in total length, sealed seam inclusive.

The seal seams are depicted on the edges as flat surface with an approximate thickness depth of 1 to 3 thousands of an inch.

This measure is irrelevant and omitted from the drawing.

The sealed products are depicted in approximately two “2” bulge with rounded surface leading onto the sealed edge.

The total depth measure of the entire packages two bulge is approximately 1″ one inch.

The product is flexible plastic and may be folded in half for smaller storage spaces.

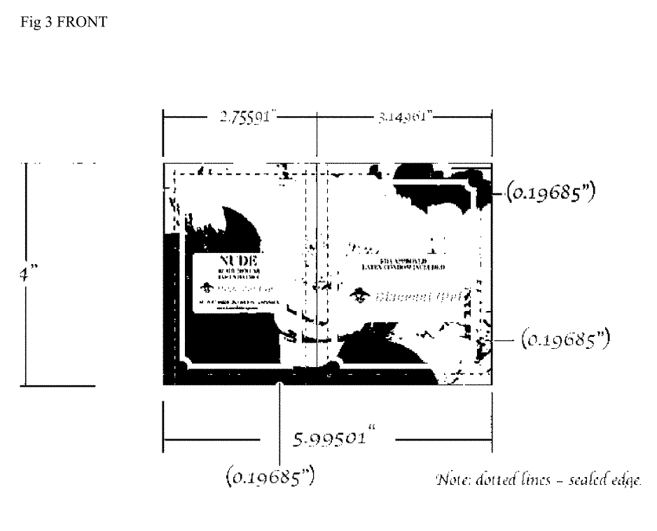

FIG. 3 Color Rendition

FIG. 3 “three” depicts an artist color rendition of drawing the drawing approximately 1″one product.

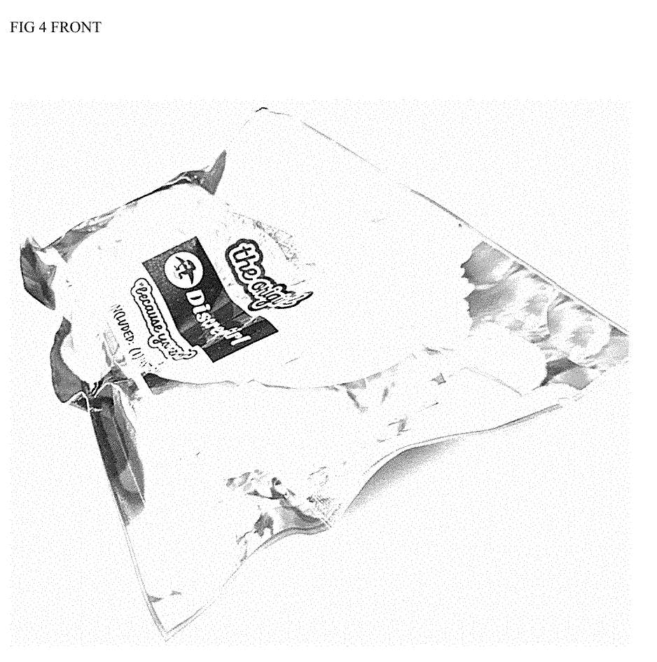

FIG. 4 Charcoal Rendition

FIG. 4 “four” depicts a charcoal rendition of the three-dimensional product packaging.

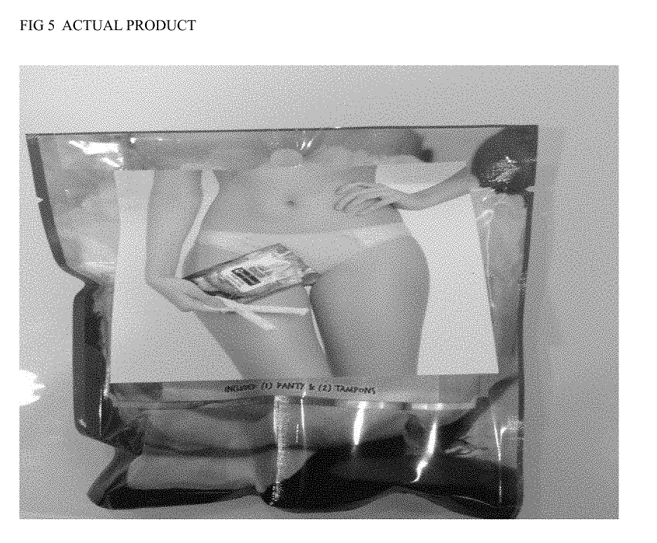

FIG. 5 Actual Product

FIG. 5 depicts the actual product photograph with an affixed label.

Note: Additional drawings are not necessary considering the simple nature of this product packaging.

Label:

Label measurement are not included as this will change with design, however the depicted label measures are approximately 1″×1″ inch and 1″×1.5″ inch inclusive.

What is claimed is:

1. An original designed plastic package assembly of packaged hygiene/safe sex kit that is discreetly and inconspicuously packed and more particularly packaged for underwear/discreetly and inconspicuously packed and more particularly packaged for underwear/undergarments and/or absorbent tampons, sanitary napkins, panty liner, wipes and prophylactic emergency kit for the unexpected menstrual cycle or sex.

2. The procedure for fabrication of claim 1 within said has two rolls of lightweight plastic flexible material representing each side of the package, which are decorated by ink with any applicable feminine design, such as flowery or plain white.

3. The parallel rolls of claim 2 within said are fed through existing machinery which will heat seal both mating surface area with a 0.19″ inch seam, in an “E shape” pattern nearing and approximate perimeter measurement of 4″×5.99″ inches of both sides.

4. The procedure of claim 3 within said will create two pockets in the surface area of 4″×5.99″.

5. The women's undergarment is compacted and folded tightly; then inserted into the packaging in one pocket of claim 4 within said.

6. The insertion into pocket number 2 of claim 4 within said either a choice of; tampon, sanitary pads, or condom with hand sanitizer.

7. The package is vacuum heat sealed of claim 3 within said to enclose the products.

8. The vacuum sealed package of claim 3 within said has a center hole at the top of the sealed area that will allow such as vending via; vending machines, store racks, and/or any retail display.

9. The vacuum packed sealed plastic packaging are adorned with any theme such as flowers, colors, and sealed within are of claim 1 within said has sterile scented womens undergarments and/or leggings and/or feminine sterile hygienic products, wipes and/or condoms.
If the unexpected menstrual happens or leakage from incontinence she will have the necessary feminine hygiene items on hand discreetly packaged.

10. The packaged condom with the undergarments allows to be prepared for safe sex with a discreet packaged of claim 9.

11. The utilitarian product that is a conveniently packaged emergency kit for females of various ages to ensure that the necessary clean sterile undergarment and feminine hygiene items are on had when the unexpected menstrual happens within said of claim 1.

12. The females menstrual cycle makes it necessary of claim 9 within said for the females to have on hand or availability of feminine hygiene products such as tampons, sanitary napkins, panty liners, wipes and clean undergarments because females never really know the exact day their period will start.
The product is absolutely inconspicuously of claim 1 within said, because of its packaging. if the product falls out into public view and someone sees it, it will save them of the embarrassment.

13. The product is absolutely inconspicuously of claim 1 within said, because of its packaging. if the product falls out into public view and someone sees it, it will save them of the embarrassment.

14. The invention is ideal to keep on hand of claim 1 within said for the unexpected period, because females just never really know when will be onset of her period. Therefore, this a great solution for females of various ages for their period or leakage and/or safe sex.
